(19) United States
(12) Patent Application Publication
(10) Pub. No.: US 2006/0088490 A1
(43) Pub. Date: Apr. 27, 2006

(54) Method for treating hair

(75) Inventors: Ryo Maruyama, Osaka (JP); Satoshi Uzue, Osaka (JP); Koki Kashihara, Osaka (JP); Kengo Maeyama, Osaka (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Milbon Co., Ltd., Osaka (JP)

(21) Appl. No.: 11/181,920

(22) Filed: Jul. 15, 2005

(30) Foreign Application Priority Data
Oct. 21, 2004 (JP) 2004-306440
Oct. 26, 2004 (JP) 2004-310292
Oct. 29, 2004 (JP) 2004-314956
Mar. 30, 2005 (JP) 2005-97916
May 30, 2005 (JP) 2005-156839

(51) Int. Cl. A61K 8/898

(52) U.S. Cl. 424/70.12; 424/70.31

(57) ABSTRACT

The present invention provides a treating method having a good continuity of the reform effects of the hair, and which is also excellent in safety. In the method, a hair inside repair agent including at least water and a component to repair an inside of the hair is applied as a step (A). A nonaqueous pretreatment agent including at least a methyl hydrogenpolysiloxane, a polyoxyethylene polyoxypropylene alkylether, and an acid is applied to the hair as step (B). A hair coating agent including at least an amino functional silicone is applied to the hair as step (C).

### TECHNICAL FIELD OF THE INVENTION

The present invention relates to a method for treating hair.

### TECHNICAL BACKGROUND

Conventionally, for the purpose of improving the flexibility and the properties of hair combing, so-called hair conditioning agent has been used and applied. Generally, a hair conditioning agent includes a cationic surfactant as a main component, which improves the flexibility and the property for combing the hair. Such a conventional hair conditioner also includes fats and oils, fatty alcohols, silicone oils, and polyhydric alcohols, etc., for the purpose of protection of the hair surface, provision of hair sheen, and improvement of the properties for hair combing.

However, recent spread of the permanent wave and hair dye gives much damage to the hair, so that the conventional hair conditioning agents can not be fully expected to improve the properties of the hair. Under the situations, one technique has been proposed, which uses methylhydrogen polysiloxane (INCI: methicone) to treat hair, as disclosed in Japanese Laid Open Patent Publication No. 2003-81784.

The methylhydrogen polysiloxane as disclosed has a hydrophilic powder whose surface is made hydrophobic to give hydrophobic and water resistance properties. For example, when such powders are incorporated into cosmetics, the powders prevent aggregation therebetween. The art disclosed in the patent reference is based on the phenomenon that the methylhydrogen polysiloxane can be combined with the OH groups and SH groups in the hair, and also can be combined with the hair conditioning agent. By this reason, the methylhydrogen polysiloxane is included in a pretreatment agent, which is used before the hair treatment, so as to improve the properties such as flexibility and the easiness in combing the hair.

It has been also known that an amino functional silicone is included in a hair conditioning agent in order to make the dried hair soft and moist. For example, Japanese Laid Open Patent Publication No. 2004-10581 discloses a hair conditioning composition including an amino functional silicone together with a silicone having a low viscosity at a specific ratio, which further includes a cationic surfactant, a betaine type amphoteric surfactant and a fatty alcohol.

Further, another art is known to improve the continuity of the hair reform effects based on the hair conditioning agent. The method as known uses a first agent, a second agent and a third agent. That is, this method comprises applying to the hair a first agent including an amino functional silicone having a specific structure; applying to the hair a second agent which is nonaqueous and includes a methylhydrogen polysiloxane having a specific structure; and applying to the hair a third agent including an amino functional silicone having a specific structure. See Japanese Laid Open Patent Publication No. 2001-226236.

### SUMMARY OF THE INVENTION

### OBJECTIVES OF THE INVENTION

However, conventional hair conditioning agents, even though it is applied to the hair, lose its reform effects upon washing the hair. Therefore, the hair is generally treated every time when it is washed. Thus, it has been recently demanded to develop a hair conditioning agent whose hair reform effects can be continued even after the hair washing is repeated several times. The art disclosed in Japanese Laid Open Patent Publications Nos. 2003-81784 and 2004-10581 do not fully disclose or suggest the continuity of the hair reform effects in case of washing the hair.

Also, the art disclosed in Japanese Laid Open Patent Publication No. 2001-226236 focuses on the improvement in the continuity of the hair reform effects by using a metylhydrogen polysiloxane. However, upon using the agent, the disclosed methylhydrogen polysiloxane would have to consider the aspects as follows:

That is, the methylhydrogen polysiloxane reacts with water to generate hydrogen gas. Thus, a hair treatment agent including a methylhydrogen polysiloxane generates hydrogen gas as the day goes on, which accompanies with the issues of the stability and safety.

Also, the methylhydrogen polysiloxane is used in a nonaqueous solution as a solvent such as lower alcohols including ethanol and isopropanol, or ester oils including isononyl isononanoate, in view of the solubility and penetration into the hair. However, when the lower alcohols such as ethanol are included as a solvent, and if the solution includes the lower alcohols at a high content, the resulting agent gives strong irritation to the scalp and/or accelerates color fading of the hair dye. On the other hand, if oils such as the ester oils are included at a high content, the treated hair will appear greasy. Thus, the effects of the methylhydrogen polysiloxane have not been fully educed.

In view of the above, the present invention provides a method for treating hair by which the hair reform effects such as improvement of the flexibility and the smooth feeling of the hair are continued, also being excellent in safety.

### MEANS FOR SOLVING THE OBJECTIVES

The objectives have been accomplished by the present invention. The present invention provides a method for treating hair, characterized in that a hair inside repair agent is applied to the hair, which includes at least water and a component to repair the inside of the hair [step (A)]; after step (A), a nonaqueous pretreatment agent including at least a methylhydrogen polysiloxane, a polyoxyethylene polyoxypropylene alkylether represented by the following formula (1), and an acid is applied to the hair [step (B)]; and after step (B), a hair coating agent including at least an amino functional silicone is applied to the hair [step (C)]. 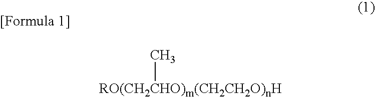
In the formula (1), “R” represents an alkyl group or alkenyl group having a carbon number of 1 to 24, in which one or more hydrogen atom thereof can be replaced with a substituent, and “m” and “n” represents 1≦m≦20 and 1≦n≦8, respectively.

The treatment method of the present invention repairs the inside of the hair by the three steps, that is, step (A), step (B) and step (C). This method also forms a film having hair reform effects on the hair surface. Thus, the method can reform the hair both from the inside of the hair and from the surface of the hair. Also, the surface of the hair, whose inside is repaired, is covered by the film which continues the effects. Further, the pretreatment agent according to the present invention including a methylhydrogen polysiloxane gives less irritation to the body. Also, the methylhydrogen polysiloxane is inhibited to react with water during storage of the pretreatment agent. Thus, the method of the present invention can fully educe the effects of the methylhydrogen polysiloxane, and also, is excellent in safety.

As explained above, the steps (A) to (C) form a film having reform effects on the surface of the treated hair. The film is derived from the amino functional silicone which is included in the hair coating agent used in step (C). The forming of the film is linked to the methylhydrogen polysiloxane included in the pretreatment agent used in step (B). Also, when a part of the film is broken in the dairy life and the effects are deteriorated, it is possible to apply the film repair agent including at least an amino functional silicone so as to repair the broken portions of the film. Such process for repairing can be carried out, after step (C), as step (D), to apply a film repair agent, which includes at least an amino functional silicone so as to repair the broken portion of the film derived from the amino functional silicone formed on the hair surface. By step (D), the deterioration of the reform effects by the film is restricted, thereby continuing the effects.

### EFFECTS OF THE INVENTION

According to the present invention, the hair treating method shows the good continuity of the hair reform effects, and also is found to be excellent in safety.

### BEST MODE OF THE INVENTION

The method for treating hair of the present invention is described based on the steps, respectively. First of all, the hair inside repair agent is applied to the hair such that the components of the hair inside repair agent are penetrated inside the hair, so as to repair the inside of the hair [Step (A)]. It should be noted that the hair to be subjected to the hair inside repair can be preferably washed with shampoo, and then washed with water before step (A).

The hair inside repair agent used in step (A) includes water and a component to repair the hair inside. Thus, the hair inside repair agent is applied to the hair such that the water and the component to repair the inside of the hair are fully penetrated inside the hair so as to repair the hair inside.

In the hair inside repair agent of the present invention, the component to repair the hair inside is not limited, and can be used by selecting from various components which are used in the known hair conditioning agents. Specific components can be listed, which include oil components such as fats and oils, waxes, fatty alcohols, ester oils and hydrocarbons, for the purpose of supplying oil contents to the hair; a component such as polypeptides to repair the damage of the hair protein; a components such as basic amino acids acting as a water retention component in the hair; and a component such as cholesterols and ceramides to restrict the release of the moisture inside the hair.

The fats and oils can include caster oil, olive oil, camellia oil, macademia nut oil, grape seed oil, mink oil, yolk oil, cacao butter, coconut butter, palm oil, japan wax and avocado oil. The waxes can include beeswax, lanolin, carnauba wax and candelilla wax. The fatty alcohols can include lauryl alcohol, cetanol, cetostearyl alcohol, stearyl alcohol, oleyl alcohol, behenyl alcohol, lanolin alcohol and octyl dodecanol. The ester oils can include di-2-ethylhexyl succinate, isocetyl isostearate, cholesteryl isostearate, cetyl octanoate, octyldodecyl oleate, isocetyl stearate, cholesteryl stearate, cetyl lactate, isopropyl myristate, myristyl myristate, octyldodecyl myristate, glyceryl myristate and polyglyceryl monomyristate. The hydrocarbons can include liquid paraffin, light liquid isoparaffin, liquid isoparaffin, squalane, petrolatum and microcrystalline wax.

The polypeptides can include keratin protein derivatives such as hydrolyzed keratin; collagen protein derivatives such as hydrolyzed collagen; silk protein derivatives; and soybean protein derivatives, which are vegetable protein derivatives, including hydrolyzed vegetable protein propylene glycol (PG)-propylsilanetriol and so on. The basic amino acids can include L-arginine and so on. The ceramides can include ceramide 1, ceramide 2, ceramide 3, ceramide 4, ceramide 5 and ceramide 6.

In the hair inside repair agent of the present invention, it is preferable to include a component to adjust the hair surface evenly, including an anionic polymer such as a polyacrylamide, and a phospholipid polymer such as 2-methacryloyloxyethyl phosphoryl choline 2-hydroxy-3-methacryloyloxypropyltrimethyl ammonium chloride copolymer. Also, the hair inside repair agent preferably includes a component to form a film on the hair surface for water retention to restrict the release of the moisture in the hair to the outside, including a sucrose fatty acid esters such as sucrose laurate.

Also, if the hair to be treated is fine or average hair, and if the hair is not seriously damaged, the hair inside repair agent can preferably include an amino functional silicone and a polyoxyalkylene benzyl ether.

In the specification, the terms “amino functional silicone” means a compound having a main chain of a silicone and a side chain of an amino group. The amino functional silicone used in the hair inside repair agent can include aminoethyl aminopropyl siloxane dimethylsiloxane copolymer, such as the one represented by the following formula (2); and aminoethyl aminopropylmethyl polysiloxane dimethyl polysiloxane copolymer such as the one represented by the following formula (3). These amino functional silicones can form a film on the hair surface, which is strongly resistant to washing. Thus, the effects such as the easiness in combing the hair, soft feeling and sheen can be continued. Also, the damage to the hair by the dryer can be protected.

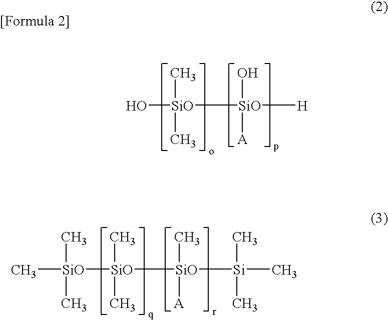

In the formula (2), “A” represents —(CH₂)₃NH(CH₂)₂NH₂, and the variations “o” and “p” represent as follows: o>1 and p>1 with o+p>10. In the formula (3), “A” represents the same as the formula (2), and the variations “q” and “r” represent as follows: q>1 and r>1 with q+r>10.

The commercial products of the aminoethylaminopropyl siloxane dimethylsiloxane copolymer can be listed as “SM8704C” manufactured by Dow Corning Toray Co., Ltd. and “XF42-C0330” manufactured by GE Toshiba Silicones Co., Ltd. The commercial product of the aminoethylaminopropylmethylpolysiloxane dimethylpolysiloxane copolymer can be listed as “SF8451C” and “SF8452C” manufactured by Dow Corning Toray Co., Ltd. These amino functional silicones can be used alone or in combination.

As the above mentioned polyoxyalkylene benzyl ether, the ones can be used in which benzyl alcohol is added to ethylene oxide at a mole ratio of 1:2, based on the averaged number of moles, and/or in which benzyl alcohol is added to ethylene oxide and propylene oxide at a mole ratio of 1:2:1, based on the averaged number of moles.

According to the present invention, polyoxyalkylene benzyl ether together with an amino functional silicone is provided in the hair inside repair agent. The amino functional silicone should be applied to the hair from the tip to the middle without application to the root, and thereby the greasy feelings and unevenness of the gloss can be avoided. As the polyoxyalkylene benzyl ether of the present invention, the ones can be used in which benzyl alcohol is added to ethylene oxide at a mole ratio of 1:2 based on an averaged number of moles, [which is hereinafter referred to as “polyoxyethylene (2E.O.) benzyl ether,” the symbol “(2E.O)” showing an averaged number of moles of the ethylene oxide addition]; and in which benzyl alcohol is added to ethylene oxide and propylene oxide at a mole ratio of 1:2:1 based on an averaged number of moles [, which is hereinafter referred to as “polyoxyethylene (2E.O.) polyoxypropylene (1P.O) benzyl ether, the symbol “(2E.O.)” showing an averaged number of moles of the ethylene oxide addition, and (1P.O.) showing an averaged number of moles of the propylene oxide addition]. A typical structure of the polyoxyethylene (2E.O.) benzyl ether has the following formula (4). A typical structure of the polyoxyethylene (2E.O.) polyoxypropylene (1P.O.) benzyl ether has the following formula (5).

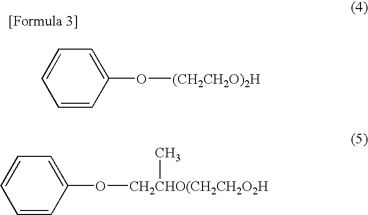

In the above, the polyoxyethylene (2E.O.) benzyl ether and the polyoxyethylene (2E.O) polyoxypropylene (1P.O) benzyl ether are described by the preparation method. However, the method for the preparation is not limited in the present invention. So long as the resulting compound of the polyoxyethylene (2E.O.) benzyl ether has a structure in which benzyl alcohol is added to ethylene oxide at a molar ratio of 1:2 based on the averaged number of moles, and so long as the resulting compound of the polyoxyethylene (2E.O) polyoxypropylene (1P.O) benzyl ether has a structure in which benzyl ether is added to ethylene oxide and propylene oxide at a molar ratio of 1:2:1 based on the averaged number of moles, such compounds are acceptable in the present invention. The polyoxyalkylene benzyl ether can be used alone or in combination thereof.

The amino functional silicone and the polyalkylene benzyl ether can be included in a hair inside repair agent which is used to treat hair, being fine or average hair, not seriously damaged.

Further, if the hair to be treated is relatively hard, frizzy (so-called frizzy hair) or waved by the treatment of a permanent wave agent, the hair inside repair agent can include the anionic polymers such as a polyacrylamide, and also, the hair inside repair agent is adjusted to have a pH value of 5 or less. When the hair inside repair agent including the anionic polymer is adjusted to have a pH value of 5 or less, the amount of the anionic polymer is increased where the agent is attached to the ends of the hair, which is particularly apt to be damaged. The anionic polymer included in the hair inside repair agent is adsorbed to the hair surface. In this respect, when a cationic compound such as a cationic polymer and a cationic surfactant is included in the hair coating agent used in step (C), an interaction between the anionic polymer and the cationic compound occur to improve adhesion of the cationic compound to the hair surface, thereby advantageously increasing the soft feelings of the hair. For this purpose, the hair inside repair agent can preferably have a pH value of 4 or less, and more preferably of 3 or less, and further preferably of 2 or more.

If the pH value of the hair inside repair agent becomes out of the range as described above, a pH adjuster can be added in the hair inside repair agent. The pH adjuster used in the hair inside repair agent can include an organic acid such as citric acid, glycolic acid, succinic acid, tartaric acid, lactic acid, fumaric acid, malic acid, levulinic acid, butyric acid, oxalic acid and maleic acid; an inorganic acid such as phosphoric acid, hydrochloric acid, sulfuric acid and nitric acid; a chelate agent such as trisodium hydroxyethyl ethylenediamine triacetate, edetic acid and the salt thereof, 1-hydroxyethane-1,1-diphosphonic acid and the salt thereof. Among these compounds, phosphoric acid, citric acid, and hydroxyethane diphosphonic acid can be used preferably.

In addition, the hair inside repair agent can preferably include various additives which have been known. These additives can include cationic polymers, silicones, cyclic polymers, moisturizers, surfactants, thickeners, preservatives, chelate agents, fragrances, alkaline chemicals, antioxidants and so on.

The cationic polymers can include cationic cellulose and cationic Guar Gum. The silicones can include dimethyl silicone, highly polymerized dimethyl silicone, cyclic silicone, and polyether modified silicone. The cyclic polymer can include decamethylcyclopentasiloxane. The moisturizers can include glycerin, diglycerin, propylene glycol, 1,3-butylene glycol, dipropylene glycol, sodium pyrrolidone carboxylate, hyaluronic acid, sorbitol, and highly polymerized polyethylene glycol. The surfactant can include cationic surfactants such as alkyltrimethyl ammonium chlorides such as lauryltrimethyl ammonium chloride, stearyltrimethyl ammonium chloride, and distearyldimethyl ammonium chloride, cetyltrimethyl ammonium bromide, stearyltrimethyl ammonium bromide and stearyltrimethyl ammonium saccharinate; anionic surfactants such as sodium lauryl sulfate, sodium polyoxyethylene laurylether sulfate, sodium methyl stearoyl taurate, triethanolamine dodecylbenzenesulfonate, sodium tetradecene sulfonate, polyoxyethylene lauryl ether phosphate, and the salt thereof; nonionic surfactants such as polyoxyethylene alkylethers including polyoxyethylene stearylether and polyoxyethylene cetylether, polyoxyethylene alkylphenylethers, polyoxyethylene polyoxypropylene alkylether, polyoxyethylene polyoxypropylene glycol, glycerin fatty acid ester, polyglycerin fatty acid ester, sorbitan fatty acid ester such as sorbitan monostearate, polyoxyethylene sorbitan monolaurate, polyoxyethylene sorbitan fatty acid ester, polyoxyalkylene benzyl ether (excluding the polyoxyalkylene benzyl ether as described above), polyoxyethylene lauryl ether (7E.O.) [(7E.O.) meaning as the same manner as (1E.O.)], polyoxyethylene castor oil, polyoxyethylene hydrogenated castor oil, sucrose fatty acid ester, alkyl polyglycoside, N-alkyl dimethyl amine oxide, and polyoxyethylene propylene glycol fatty acid ester; and amphoteric surfactants such as cocamidepropyl betaine, lauryldimethylamino acetic acid betaine, lauramidepropyl betaine and 2-alkyl-N-carboxymethyl-N-hydroxyethyl imidazolium betaine. The thickeners can include carrageenan, xanthan gum, sodium carboxy methyl cellulose, sodium alginate, polyvinyl alcohol, polyvinyl pyrrolidone, carboxyvinyl polymer and sodium polyacrylate. The preservatives can include sodium benzoate, benzalkonium chloride, sorbic acid, potassium sorbate, methyl paraoxybenzoate, ethyl paraohydroxybenzoate, propyl parahydroxybenzoate, butyl parahydroxybenzoate and 2-phenoxyethanol.

The chelate agents can include edetic acid, disodium edetate, citric acid, tartaric acid, salicylic acid and 1-hydroxy-1,1-diphosphonic acid.

The antioxidants can include vitamin E (d-δ-tocopherol, etc.), d-δ-tocopherol acetate, and etc.

The hair inside repair agent can be prepared in the form of cream, lotion, gel and so on, including water as a main disperse medium. A part of the components thereof can be solved in the water. Also, the disperse medium can be water alone, or including an organic solvent of a lower alcohol such as ethanol and isopropanol. It should be noted that the increase of the organic solvent content in the water will give much irritation to the scalp, so that the organic solvent is preferably included at an amount of 5 mass % or less.

The hair inside repair agent can be used, which has various compositions in accordance with the condition of the hair to be treated. For example, in case where the hair is fine or average hair, and if the hair is not much damaged, it is preferable to use a hair inside repair agent including water as a disperse medium, the protein derivative derived from a vegetable at an amount of 0.01 to 1 mass %; the ceramides and/or cholesterol at an amount of 0.001 to 0.1 mass %; the basic amino acids at an amount of 0.01 to 1 mass %; the phospholipid polymer at an amount of 0.01 to 1 mass %; the cationic surfactant at an amount of 0.5 to 5 mass %; the fatty alcohols at an amount of 1 to 10 mass %; and the amino functional silicone at an amount of 0.1 to 3 mass %. In addition to the components as described above, the hair inside repair agent can include the components as listed before, if necessary.

On the other hand, the following formula can be used for the hair being relatively hard, frizzy (so-called frizzy hair) or waved by the treatment of a permanent wave agent: a hair inside repair agent including water as a disperse medium; the keratin derivatives at an amount of 0.1 to 1 mass %; the ceramides and/or cholesterol at an amount of 0.001 to 0.1 mass %; the anionic polymer at an amount of 0.1 to 5 mass %; the sucrose fatty acid ester at an amount of 0.1 to 5 mass %; the fatty alcohols at an amount of 0.1 to 5 mass %; and the amino functional silicone at an amount of 0.1 to 5 mass %. The pH adjuster can be added to adjust the pH value within the range as descried above. In addition to the components as described above, the hair inside repair agent can include the components as listed before, if necessary.

The amount of the hair inside repair agent depends on the amount of the hair to be treated. For example, when treating shoulder-length hair (hair having a length to touch the shoulder), it can be preferably applied at an amount of 10 to 20 g. After the hair inside repair agent is applied, the step (B) can be carried out without a period, and alternatively, the step (B) can be carried out after waiting for 5 to 10 minutes.

Then, the hair surface, after the application of the hair inside repair agent, is subjected to application of the pretreatment agent of the step (B). The pretreatment agent used in step (B) includes a methylhydrogen polysiloxane, which chemically combines with the amino functional silicone included in the hair coating agent used in step (C), so as to form a film on the hair surface. Thus, the pretreatment agent improves attachment of the film to the hair surface. In other words, step (B) is a step to be carried out to form a foundation for the film.

The pretreatment agent used in step (B) includes at least a methylhydrogen polysiloxane, a polyoxyethylene polyoxypropylene alkylether as represented by the formula (1), and an acid.

The methylhydrogen polysiloxane is, as described above, a component to improve the adhesion of the film to the hair surface, the film being formed by the amino functional silicone used in step (C). Further, the methylhydrogen polysiloxane can give an excellent flexibility and properties for combing the hair. It should be noted that the methylhydrogen polysiloxane is a methylpolysiloxane in which a part of the methyl groups is substituted with hydrogen. The commercial products can include “SH1107C” manufactured by Dow Corning Toray Co., Ltd., and “KF-99” manufactured by Shin-Etsu Chemical Co. Ltd.

The polyoxyethylene polyoxypropylene alkylether and the acid included in the pretreatment agent are components to improve the safety and temporal stability of the pretreatment agent, which restrict the reaction between the methylhydrogen polysiloxane and water to generate hydrogen gas. Also, the coexistence of the polyoxyethylene polyoxypropylene alkylether with the methylhydrogen polysiloxane further enhances the effects to improve the flexibility and the easiness in combing the hair, which is derived from the methylhydrogen polysiloxane.

The polyoxyethylene polyoxypropylene alkylether is used, which has a structure as represented by the formula (1). In the formula (1), “R” represents an alkyl group or alkenyl group having a carbon number of 1 to 24. One or more hydrogen atoms of the alkyl group or the alkenyl group can be replaced with a substituent such as hydroxyl group and silyl group.

The Examples of the polyoxyethylene polyoxypropylene alkylether as represented by the formula (1) can include polyoxyethylene polyoxypropylene cetylether, polyoxyethylene polyoxypropylene behenylether, polyoxyethylene polyoxypropylene 2-decyltetradecylether, polyoxyethylene polyoxypropylene butylether, polyoxyethylene polyoxypropylene hydrogenated lanolin, and polyoxyethylene polyoxypropylene glycelyl ether, in which the values of “m” and “n” fall within the ranges as described above. These compounds as listed can be used alone or in combination thereof. Among them, polyoxyethylene polyoxypropylene butylether can be preferably used. As a commercially available product, “ADEKA CARPOL MH-4” manufactured by Asahi Denka Co., Ltd. can be preferably used.

The acid in the pretreatment agent can include organic acids such as salicylic acid, citric acid, succinic acid, tartaric acid and malic acid. These compounds can be used alone or in combination thereof. Among them, salicylic acid can be preferably used in view of solubility.

Also, the pretreatment agent can include, so long as the features thereof are not adversely affected, any components other than the methylhydrogen polysiloxane, the polyoxyethylene polyoxypropylene alkylether, and the acid. These compounds can include surfactants, oils and fragrances.

The surfactants can include compounds used in the hair inside repair agent as described above. The oils can include ester oils such as isopropyl myristate, dioctyl succinate, octyldodecyl myristate, 2-ethylhexyl isononanoate; silicone oils such as dimethyl polysiloxane, methyl phenyl polysiloxane, polyether modified silicone and amino functional silicone; and fats and oils such as macademia nut oil, soybean oil, sunflower seed oil, castor oil and yolk oil.

In addition, the pretreatment agent can include antioxidants such as dibutylhydroxy toluene.

The pretreatment agent used in the step (B) can preferably have the composition as follows: The methylhydrogen polysiloxane can be included at an amount of 0.1 mass % or more and preferably at an amount of 1 mass % or more, and at an amount of 10 mass % or less and preferably at an amount of 5 mass % or less. The polyoxyethylene polyoxypropylene alkylether can be included at an amount of 1 mass % or more and preferably at an amount of 30 mass % or more, and at an amount of 80 mass % or less and preferably at an amount of 70 mass % or less. The acid can be included at an amount of 0.005 mass % or more and preferably at an amount of 0.01 mass % or more, and at an amount of 1 mass % or less and preferably at an amount of 0.1 mass % or less. Further, the pretreatment agent can be prepared to become 100 mass % of the whole components, including the surfactant, oils and fragrances. As described above, when the solvent for the methylhydrogen polysiloxane includes an organic solvent such as a lower alcohol at a high content, irritation to the scalp should be considered. On the other hand, oils such as ester oils are included at a high content, the hair results in greasy appearance. However, when the pretreatment agent includes the polyoxyethylene polyoxypropylene alkylether as represented by the formula (1) at an amount as described above, the amount of the organic solvent or ester oils can be reduced or eliminated, resulting in less irritation to the scalp and less greasy appearance, and being excellent in safety.

The pretreatment agent can be applied to the hair at an amount of 2 ml to 10 ml, for example, in case of shoulder-length hair (hair has a length to touch the shoulder). It is preferable to use the pretreatment agent such that the agent is mainly applied to the ends thereof and only applied up to the middle of the length. A film is formed in the step (C) carried out after step (B), but if the film is formed around the root of the hair, it may give greasy feelings and uneven sheen to the hair. Therefore, the pretreatment agent, improving the adhesion of the film to the hair, also should not be applied to the root of the hair, thereby the film is avoided from being formed around the root of the hair.

After applying the pretreatment agent, the hair is rubbed and combed to fully be saturated with the agent. Thereafter, the hair can be washed with water. Also, it can be washed with shampoo and then washed with water. The washing with water can improve the reactivity of the methylhydrogen polysiloxane attached to the hair surface, thereby promoting chemical reaction with the amino functional silicone included in the hair coating agent of step (C).

Then, to the hair subjected to the pretreatment agent and washed with water, the hair coating agent is applied as step (C). Step (C) forms a film derived from the amino functional silicone on the hair surface. This film gives easiness of the combing, flexibility and sheen to the hair, and improvement of the resistance to the heat by a dryer. Also, the film, formed from the amino functional silicone and the pretreatment agent applied to the hair in step (B), is resistant to the washing. Thus, the effects of the film continue.

The amino functional silicone can include compounds as represented by the formula (2) and formula (3), which are used in the hair inside repair agent of step (A).

The hair coating agent can preferably include, in addition to the amino functional silicone, various components for the purpose of reforming the hair. These components can include a component such as cyclic polymers and spherical polymers to give smooth feeling to the hair surface, a component such as ester oils to avoid the hair from tangling, a component such as lanolin fatty acid esters to avoid flyaway of the hair ends, a component such as cationic polymers to make the hair a soft feeling, and a component such as wax esters to restrict the release of the moisture from the hair.

The cyclic polymers can include decamethylcyclopentasiloxane. The spherical polymers can include a crosslinked methylpolysiloxane. The ester oils can include isodecyl neopentanoate. The lanolin fatty acid esters can include cholesteryl lanolate. The cationic polymers can include polyethyleneglycol.epichlorohydrin.coconut alkyl amine.dipropylene triamine condensation product, dimethyldiallyl ammonium chloride acrylic acid copolymer, dimethyldiallyl ammonium chloride.acrylamide copolymer, polyvinylpyrrolidone N,N-dimethyl aminoethyl methacrylic acid copolymer diethyl sulfate, cationic cellulose and cationic Guar Gum. The wax esters can include jojoba oil.

The hair coating agent can include, in addition to the amino functional silicone and the various components as described above, various additives which are known to use in a hair conditioning agent. These additives can include fats and oils; hydrocarbons; silicones (other than the amino functional silicone, the cyclic polymers and the spherical polymers as descriobed above, which is hereinafter interpreted in the same manner in the description for the hair coating agent); waxes; higher fatty acids; fatty alcohols; ester oils (other than isodecyl neopentanoate, which is hereinafter interpreted in the same manner in the description of the hair coating agent); surfactants; moisturizers; thickeners preservatives; chelate agents; and fragrances.

The examples of the fats and oils, hydrocarbons, silicones, waxes, fatty alcohols, ester oil s, surfactants, moisturizers, thickeners, preservatives and chelate agents can include the various components used in the hair inside repair agent as described above. The higher fatty acid can include isostearic acid, oleic acid, stearic acid, palmitic acid, behenic acid, myristic acid, lauric acid, lanolin fatty acid, hard lanolin fatty acid, soft lanolin fatty acid and linoleic acid.

The hair coating agent can be prepared in the form of cream, lotion and gel, including water as a main disperse medium. A part of the components as described above can be solved in the water. The disperse medium can be water alone, or include an organic solvent of a lower alcohol such as ethanol and isopropanol. It should be noted that the increase of the amount of the organic solvent may give irritation to the scalp. Thus, the organic solvent can be preferably included at an amount of 5 mass % or less in the disperse medium.

The hair coating agent can have various compositions in accordance with the condition of the hair to be treated. For example, when the hair is fine hair and not much damaged, the hair coating agent can have the following composition: water as a disperse medium; the amino functional silicone at an amount of 0.1 to 1 mass %; the cyclic polymer and/or the spherical polymer at an amount of 0.5 to 5 mass %; the ester oils such as isodecyl neopentanoate at an amount of 1 to 10 mass %; the cationic surfactant at an amount of 0.5 to 5 mass %; and the fatty alcohols at an amount of 1 to 10 mass %. In addition, the hair coating agent can include various components as described before, if necessary.

On the other hand, the hair is an average hair and is not much damaged, the following hair coating agent can be used: water as a disperse medium; the amino functional silicone at an amount of 0.1 to 3 mass %; the cyclic polymer and/or spherical polymer at an amount of 0.5 to 5 mass %; the lanolin fatty acid ester at an amount of 0.1 to 1 mass %; the ester oil such as isodecyl neopentanoate at an amount of 1 to 5 mass %; the cationic surfactants at an amount of 0.5 to 5 mass %; and fatty alcohols at an amount of 1 to 10 mass %. In addition, the hair coating agent can include various components as described before, if necessary.

Further, when the hair is relatively hard, frizzy (so-called frizzy hair) or waved by the treatment of a permanent wave agent, the following hair coating agent can be used: water as a disperse medium; the amino functional silicone at an amount of 1 to 5 mass %; the wax esters at an amount of 1 to 5 mass %; the cationic polymer at an amount of 0.01 to 1 mass %; the cationic surfactants at an amount of 0.5 to 5 mass %; and fatty alcohols at an amount of 1 to 10 mass %. In addition, the hair coating agent can include various components as described before, if necessary.

In particular, if the hair is previously treated by the hair inside repair agent including the anionic polymer (in particular, having a pH value of 5 or less), the use of the hair coating agent including a cationic compound such as the cationicpolymers and the cationic surfactants will increase the adhesion of the cationic compound to the hair surface by the interaction between the anionic polymer and the cationic compounds, thereby effectively increasing the soft feeling of the hair.

The cationic surfactants, that is, cationic compounds for the hair coating agent, can preferably include, but not to be limited thereto, quaternary ammonium salts such as the alkyltrimethyl ammonium chlorides such as lauryltrimethyl ammonium chloride and stearyltrimethyl ammonium chloride; distearyldimethyl ammonium chloride, cetyltrimethyl ammonium bromide, and stearyltrymethyl ammonium bromide. Among the quaternary ammonium salts, ones having an anion of bromine ion can be preferably used in view of the attachment to the hair, including cetyltrimethyl ammonium bromideand stearyltrimethyl ammonium bromide. Further in view of giving softness to the hair, cetyltrimethyl ammonium bromide can be used.

The hair coating agent can be preferably applied, for example, to hair shoulder length hair, at an amount of 10 to 20 g. The hair coating agent can be mainly applied to the ends of the hair, and applied up to the middle of the length. As discussed before, when the film derived from the amino functional silicone is formed around the root of the hair, the hair may appear greasy and uneven sheen.

After applying the hair coating agent, the hair is preferably rubbed to fully apply the hair coating agent to the hair. Thereafter, the hair is washed with water and dried.

Thereby processed hair has the penetrated water and other components inside the hair to become a good condition, as well as a formed film at the surface thereof derived from the amino functional silicone, which improves the feelings of the hair. Also, the film formed on the hair surface is resistant to washing, and restricts the release of the moisture inside the hair. Therefore, the treating method of the present invention continues the effects for a relatively long period.

However, the film of the treated hair formed on the hair surface is gradually broken by daily life so as to lose the effects. If the effects of the treating method are much deteriorated, the treating method of the present invention has to be repeated. However, if the film of the hair surface is partially damaged and the effects of the treating method are not much deteriorated, the hair can be subjected to step (D) as described hereinafter. In step (D), a film repair agent is applied to the hair to repair the film for recovering and maintaining the effects of the film.

The film repair agent used in step (D) can preferably use the one including an amino functional silicone, which is a material of the film formed on the hair surface. The amino functional silicone can preferably be as same as that used in the hair coating agent.

The film repair agent can preferably include, in addition to the amino functional silicone, a component including cyclic polymers such as decamethylcyclopentasiloxane to give smooth feeling to the hair surface; and a component including wax esters such as jojoba oil to cover the hair surface to restrict the release of the moisture in the hair.

Also, in addition to the amino functional silicone and the various components as described above, the film repair agent can include various additives which are known to use in a hair conditioning agent. These additives can include silicones (other than the amino functional silicone and the cyclic polymer, which is interpreted in the same manner for the description for the film repair agent,) higher fatty acids, fatty alcohols, ester oils, surfactants, moisturizers, preservatives, vegetable seed extracts and fragrances.

The examples of the silicones, higher fatty acids, fatty alcohols, ester oils, surfactants, moisturizers and preservatives can include the additives as used in the hair inside repair agent as described before. The vegetable seed extracts can include sunflower seed extract.

The film repair agent can be prepared in the form of cream, lotion and gel, including water as a main disperse medium. A part of the components can be solved in the water. The disperse medium can be water alone, or include an organic solvent of a lower alcohol such as ethanol and isopropanol. It should be noted that the increase of the organic solvent in the disperse medium may increase the irritation given to the scalp, and therefore, the organic solvent can be limited to be included in an amount of 5 mass % or less in the disperse medium.

The film repair agent can have various compositions in accordance with the condition of the hair to be treated. For example, when the hair is fine or average hair and if the hair is not much damaged, the following film repair agent can be preferably used: water as a disperse medium; the amino functional silicone at an amount of 1 to 10 mass %; the cyclic polymer at an amount of 0.5 to 5 mass %; the cationic surfactants at an amount of 0.5 to 5 mass %; and the fatty alcohols at an amount of 1 to 10 mass %. In addition, the film repair agent can include various components as described before, if necessary.

Also, when the hair is relatively hard, or frizzy (so-called frizzy hair) or waved by a permanent wave agent, the following hair coating agent can be used: water as a disperse medium; the amino functional silicones at an amount of 1 to 10%; the wax esters at an amount of 1 to 5 mass %; the cationic surfactants at an amount of 0.5 to 5 mass %; and the fatty alcohols at an amount of 1 to 10 mass %. In addition, the film repair agent can include various components as described before, if necessary.

The amount of the film repair agent to be applied is not limited. For example, when the film is damaged, the film repair agent can be applied mainly at the portions losing the feelings by the film (such as flexibility and sheen) at an appropriate amount.

### EXAMPLES

The present invention is described hereinafter based on the examples. The examples described here are not construed as limiting the scope of the invention, and the present invention can be modified within the spirits of the present invention as described in this specification, which should be also construed to be covered by the scope of the present invention.

It should be noted that in the description of the examples, the symbol “%” means “mass %.” The contents of each of the hair inside repair agent, pretreatment agent, hair coating agent and film repair agent are described by “%,” and the total of the each agent becomes 100%. In the tables as described hereinafter, the contents are shown without the symbol “%.”

The following materials were used in the Examples and Comparative Examples.

(1) Hydrolyzed vegetable protein PG-propylsilanetriol: “KERAVIS” manufactured by Croda Colloids Ltd.

(2) ceramide 2: “LIQUID CRYSTA CERAMIDE 2” manufactured by Takasago International Corporation,

(3) L-arginine manufactured by Ajinomoto Co., Inc.

(4) 2-methacryloyloxyethylphosphorylcholine.2-hydroxy-3-methacryloyloxy propyltrimethyl ammonium chloride copolymer: “LIPIDURE-C” manufactured by NOF Corporation (Nihon Yushi Corporation),

(5) stearyltrimethyl ammonium bromide: “Catinal S T B-7 0” manufactured by Toho Chemical Industry Corporation,

(6) cetanol: “KALCOL 6870” manufactured by Kao Corporation,

(7) amino functional silicone 1: “XF42-C0330” (aminoethylaminopropylmethyl siloxane dimethylsiloxane copolymer) manufactured by GE Toshiba Silicones Co., Ltd.,

(8) hydrolyzed keratin: “PROMOIS WK-GB” manufactured by Seiwa Kasei Corporation,

(9) polyacrylamide: “SEPIGEL 305” manufactured by SEPPIC Corporation,

(10) sucrose fatty acid ester: “RYOTO Sugar Ester LWA-1570” manufactured by Mitsubishi-Kagaku Foods Corporation,

(11) amino functional silicone 2: “XF49-C1109” (aminoethylaminopropylmethylsiloxane.dimethylsiloxane copolymer) manufactured by GE Toshiba Silicones Co., Ltd.

(12) methylhydrogen polysiloxane: “SH1107C” manufactured by Dow Corning Toray Co., Ltd.,

(13) polyoxyethylene polyoxypropylene butyl ether: “ADEKA CARPOL MH-4” manufactured by Asahi Denka Co., Ltd.,

(14) 2-ethylhexyl isononanoate: “ES108109” manufactured by Kokyu Alcohol Kogyo Co., Ltd.

(15) amino functional silicone 3: “KF-8005” (aminoethylaminopropylmethylsiloxane dimethylsiloxane copolymer) manufactured by Shin-Etsu Chemical Co., Ltd.,

(16) crosslinked methylpolysiloxane: “KSG-15” (spherical polymer) manufactured by Shin-Etsu Chemical Co., Ltd.,

(17) decamethylcyclopentasiloxane: “XF49-A3818” (cyclic polymer) manufactured by GE Toshiba Silicones Co., Ltd.,

(18) isodecile neopentanoate: “DUB VCI-10” manufactured by Nippon Fine Chemical Corporation,

(19) cetyltrimethyl ammonium bromide: “Catinal H T B-7 0 E T” manufactured by Toho Chemical Industry Corporation,

(20) stearyl alcohol: “KALCOL 8098” manufactured by Kao Corporation,

(21) lanolin fatty acid ester: “YOFCO CLE-S” manufactured by Nippon Fine Chemical Corporation,

(22) jojoba oil: manufactured by Koei Kogyo Co., Ltd.

(23) polyethyleneglycol.epichlorohydrin.coconut alkyl amine.dipropylene triamine condensation product: “POLYQUART H81” manufactured by Cognis Japan Ltd.

<Preparation of the Hair Inside Repair Agent>

The materials were mixed in accordance with the compositions as shown in Table 1 and Table 2, to prepare two kinds of the hair inside repair agents.

TABLE 1

| Ingredients | Content (%) |
|---|---|
| Hydrolyzed vegetable protein PG- | 0.1 |
| propylsilanetriol | |
| ceramide 2 | 0.01 |
| L-arginine | 0.5 |
| 2-methacryloyloxyethylphosphorylcholine.2- | 0.1 |
| hydroxy-3-methacryloyloxy propyltrimethyl | |
| ammonium chloride copolymer | |
| stearyltrimethyl ammonium bromide | 2.0 |
| cetanol | 5.0 |
| amino functional silicone 1 | 1.0 |
| purified water | 100 in total |

TABLE 2

| Ingredients | Content (%) |
|---|---|
| hydrolyzed keratin | 0.5 |
| ceramide 2 | 0.1 |
| polyacrylamide | 1.0 |
| sucrose fatty acid ester | 1.0 |
| amino functional silicone 2 | 1.5 |
| purified water | 100 in total |

<Preparation of the Pretreatment Agent>

The materials were mixed in accordance with the compositions as shown in Table 3 to prepare a pretreatment agent.

TABLE 3

| Ingredients | Content (%) |
|---|---|
| methylhydrogen polysiloxane | 4.0 |
| polyoxyethylene polyoxypropylene butyl | 50 |
| ether | |
| salicylic acid | 0.01 |
| 2-ethyl hexyl isononanoate | 100 in total |

<Preparation of Hair Coating Agent>

The materials were mixed in accordance with the compositions as shown in Tables 4-6 to prepare three kinds of hair coating agents.

TABLE 4

| Ingredients | Content (%) |
|---|---|
| amino functional silicone 3 | 0.5 |
| crosslinked methylpolysiloxane | 0.1 |
| decamethylcyclopentasiloxane | 3.0 |
| isodecyl neopentanoate | 3.0 |
| cetyltrimethylammonium bromide | 2.5 |
| stearyl alcohol | 5.0 |
| purified water | 100 in total |

TABLE 5

| Ingredients | Content (%) |
|---|---|
| amino functional silicone 3 | 1.0 |
| decamethylcyclopentasiloxane | 2.0 |
| lanolin fatty acid ester | 0.2 |
| isodecyl neopentanoate | 2.0 |
| cetyltrimethylammonium bromide | 2.0 |
| stearyl alcohol | 5.0 |
| purified water | 100 in total |

TABLE 6

| Ingredients | Content (%) |
|---|---|
| amino functional silicone 3 | 3.5 |
| jojoba oil | 3.5 |
| polyethyleneglycol.epichlorohydrin. | 0.1 |
| coconut alkyl amine.dipropylene tryamine | |
| condensation product | |
| cetyltrimethyl ammonium bromide | 2.0 |
| stearyl alcohol | 5.0 |
| purified water | 100 in total |

<Preparation of the Film Repair Agent>

The materials were mixed in accordance with the compositons as shown in Table 7 and Table 8 to prepare two kinds of the film repair agent.

TABLE 7

| Ingredients | Content (%) |
|---|---|
| amino functional silicone 3 | 3.0 |
| decamethylcyclopentasiloxane | 2.0 |
| cetyltrimethyl ammonium bromide | 2.0 |
| stearyl alcohol | 5.0 |
| purified water | 100 in total |

TABLE 8

| Ingredients | Content (%) |
|---|---|
| amino functional silicone 3 | 2.0 |
| jojoba oil | 3.0 |
| cetyltrimethyl ammonium bromide | 2.0 |
| stearyl alcohol | 5.0 |
| purified water | 100 in total |

It should be noted that in Tables 1, 2 and 4 to 8, “100 in total” noted in the row of the purified water means that the purified water was added into the other compositions of the hair inside repair agent, hair coating agent or film repair agent to reach 100% in total. Also, in Table 3, “100 in total” noted in the row of 2-ethylhexyl isononanoate means that 2-ethylhexyl isononanoate was added into the other compositions of the pretreatment agent to reach 100% in total.

### Example 1

To fine hair not seriously damaged, the hair inside repair agent as shown in Table 1 was applied [Step (A)]. Further, the hair after applying the hair inside repair agent is subjected to application of the pretreatment agent as shown in Table 3 [step (B)], and subsequently, the hair after applying the pretreatment agent was subjected to application of the hair coating agent as shown in Table 4 [step (C)]. In details, the following processes were carried out to treat the hair. A bundle of fine hair not seriously damaged, having a weight of 1.0 g and a length of 20 cm, was collected from a person, which was then washed with water such that the bundle wholly got wet, and then wiped with a towel to remove the excess water. Thereafter, the hair bundle is subjected to application of the hair inside repair agent at an amount of 0.5 g, and thereon the pretreatment agent was applied at an amount of 0.2 mL. Then, the hair bundle was subjected to application of the hair coating agent at an amount of 0.5 g, followed by fully washing with water and dried to complete the treatment. Then, thereby treated hair bundle was subjected to five sets of washing with shampoo and drying.

### Example 2

Hair was treated by the steps (A), (B) and (C) similar to Example 1, which was then subjected to the repair of the broken portions of the film derived from the amino functional silicone by using the film repair agent as shown in Table 7 [step (D)]. In details, the hair was carried out as follows: After carrying out the step (C), the hair bundle was subjected to ten sets of a washing step with shampoo and a drying step with a dryer. Thereafter, the hair bundle was subjected to application of the film repair agent at an amount of 0.5 g, followed by drying it to complete the treatment. Then, thereby treated hair bundle was subjected to five sets of washing with shampoo and drying.

### Comparative Example 1

Hair was treated in the same manner as Example 1 except eliminating the step (A). In other words, the hair inside repair agent was not applied to the hair. Then, thereby treated hair bundle was subjected to five sets of washing with shampoo and drying.

### Comparative Example 2

Hair was treated in the same manner as Example 2 except eliminating the step (B). In other words, the pretreatment agent was not applied to the hair. Then, thereby treated hair bundle was subjected to five sets of washing with shampoo and drying.

### Comparative Example 3

Hair was treated in the same manner as Example 1 except eliminating the step (C). In other words, the hair coating agent was not applied to the hair. Then, thereby treated hair bundle was subjected to five sets of washing with shampoo and drying.

### Example 3

To average hair not seriously damaged, the hair inside repair agent as shown in Table 1 was applied [step (A)]. After the application of the hair inside repair agent, the hair was subjected to application of the pretreatment agent as shown in Table 3 [step (B)]. Then, to the hair applying the pretreatment agent, the hair coating agent as shown in Table 5 was applied [step (C)]. The hair inside repair agent, the pretreatment agent and the hair coating agent were applied to the hair in the same manner as Example 1. Then, thereby treated hair bundle was subjected to five sets of washing with shampoo and drying.

### Example 4

To the hair treated by the steps (A), (B) and (C) in the same manner as Example 3, the film repair agent as shown in Table 7 was applied to repair the broken portions in the film derived from the amino functional silicone [step (D)]. The repairing method of the film formed on the hair surface was carried out in the same manner as Example 2. Then, thereby treated hair bundle was subjected to five sets of washing with shampoo and drying.

### Comparative Example 4

Hair was treated in the same manner as Example 3 except eliminating the step (A). In other words, the hair inside repair agent was not applied to the hair. Then, thereby treated hair bundle was subjected to five sets of washing with shampoo and drying.

### Comparative Example 5

Hair was treated in the same manner as Example 4 except eliminating the step (B). In other words, the pretreatment agent was not applied to the hair. Then, thereby treated hair bundle was subjected to five sets of washing with shampoo and drying.

### Comparative Example 6

Hair was treated in the same manner as Example 3 except eliminating the step (C). In other words, the hair coating agent was not applied to the hair. Then, thereby treated hair bundle was subjected to five sets of washing with shampoo and drying.

### Example 5

To hard and frizzy hair, the hair inside repair agent as shown in Table 2 was applied [step (A)]. Further, the hair applying the hair inside repair agent was subjected to application of the pretreatment agent as shown in Table 3 [step (B)]. Thereafter, the hair applying the pretreatment agent was subjected to application of the hair coating agent as shown in Table 6 [step (C)]. The application of the hair inside repair agent, the pretreatment agent and the hair coating agent to the hair was the same manner as Example 1. Then, thereby treated hair bundle was subjected to five sets of washing with shampoo and drying.

### Example 6

The hair was subjected to the steps (A), (B) and (C) like Example 5. Then, the film repair agent as shown in Table 8 was applied to the hair, for repairing the broken portions of the film derived from the amino functional silicone [step (D)]. The repairing method of the film formed on the hair surface was carried out in the same manner as Example 2, by using the film repair agent. Then, thereby treated hair bundle was subjected to five sets of washing with shampoo and drying.

### Comparative Example 7

Hair was treated in the same manner as Example 5 except eliminating the step (A). In other words, the hair inside repair agent was not applied to the hair. Then, thereby treated hair bundle was subjected to five sets of washing with shampoo and drying.

### Comparative Example 8

Hair was treated in the same manner as Example 6 except eliminating the step (B). In other words, the pretreatment agent was not applied to the hair. Then, thereby treated hair bundle was subjected to five sets of washing with shampoo and drying.

### Comparative Example 9

Hair was treated in the same manner as Example 5 except eliminating the step (C). In other words, the hair coating agent was not applied to the hair. Then, thereby treated hair bundle was subjected to five sets of washing with shampoo and drying.

The hair bundles as treated in accordance with Examples 1-6 and Comparative Examples 1-9, including five sets of washing with shampoo and drying, were then subjected to evaluations as follows. The results are shown in Table 9.

<Softness>

Each hair bundle was evaluated by twenty examiners. According to the evaluation by the examiners, one point was given when an examiner felt the hair bundle soft, 0 point was given when the examiner felt the hair bundle hard. The points from all the examiners were summated, which were further evaluated according to the standards as described below, so as to evaluate the effects to give the softness by the Examples and Comparative Examples. The evaluations “⊚” and “∘” were considered acceptable.

⊚: The total of the points by the examiners was 16 points or more.

∘: The total of the points by the examiners was 12 points or more but less than 16 points.

Δ: The total of the points by the examiners was 8 points or more but less than 12 points.

x: The total of the points by the examiners was less than 8 points.

<Smooth Feeling of the Hair>

To the hair treated in accordance with Examples 1-6 and Comparative Examples 1-9 including five sets of washing with shampoo and drying, frictional feel analyzer (KES-SE-STP manufactured by Kato Tech Col., Ltd) was applied to measure an MIU (averaged coefficient of friction), so as to evaluate the smooth feelings of the hair/after treating the hair in accordance with the treating method of Examples and Comparative Examples. In other words, the less value of MIU shows a smoother feeling of the hair. On the other hand, the much value of MIU shows a less smooth feeling.

As a sample for measuring the MIU, ten hairs were taken out from the hair bundles as treated in accordance with Examples 1-5 and Comparative Examples 1-9 including five sets of washing with shampoo and drying, which were aligned on a slide glass with an interval of 1 mm and attached by an adhesive tape at both ends, respectively. It should be noted that the hairs were collected such that the diameter of collected hairs was as equal as possible.

TABLE 9

| | Softness | MIU (Smooth feeling) |
|---|---|---|
| Example 1 | ⊚ | 0.291 |
| Example 2 | ⊚ | 0.278 |
| Comparative Example 1 | X | 0.511 |
| Comparative Example 2 | Δ | 0.482 |
| Comparative Example 3 | Δ | 0.427 |
| Example 3 | ⊚ | 0.280 |
| Example 4 | ⊚ | 0.273 |
| Comparative Example 4 | X | 0.494 |
| Comparative Example 5 | Δ | 0.431 |
| Comparative Example 6 | Δ | 0.403 |
| Example 5 | ⊚ | 0.296 |
| Example 6 | ⊚ | 0.283 |
| Comparative Example 7 | X | 0.499 |
| Comparative Example 8 | X | 0.477 |
| Comparative Example 9 | X | 0.419 |

Table 9 shows the followings: Examples 1 and 2, and Comparative Examples 1 to 3 were conducted against relatively fine hair not seriously damaged. Example 1, subjecting to the steps (A), (B) and (C), and Example 2, further subjecting to the step (D), showed good smooth. Feelings. On the other hand, Comparative Example 1 to 3, in which one of the steps (A), (B) and (C) was eliminated, were inferior to the treatment in accordance with Examples 1 and 2 in view of the softness and the smooth feelings.

Also, Examples 3 and 4 and Comparative Examples 4 to 6 were conducted against an average hair not seriously damaged, and Examples 5 and 6 and Comparative Examples 7 to 9 were conducted against the hard and frizzy hair. Examples 3 and 5, subjecting to the steps (A), (B) and (C), and Examples 4 and 6, further subjecting to the step (D), showed the good softness and the good smooth feelings in combing the hair by fingers. On the other hand Comparative Example 4 to 9, in which one of the steps (A), (B) and (C) was eliminated, were inferior to the treatment in accordance with Examples 3 to 6 in view of the softness and the smooth feelings.

Although the pretreatment agent of the present invention includes a highly reactive methylhydrogen polysiloxane, the other components, that is, the polyoxyethylene polyoxypropylene alkylether and the acid, restrict the reaction of the methylhydrogen polysiloxane with water to generate hydrogen gas, resulting in assuring sufficient temporal stability. The hair as treated in accordance with Examples 1-6, using the pretreatment agent with such temporal stability, had maintained the flexibility and the smooth feeling of the hair even after five repetition of the washing, being good in continuity of the hair reform effects such as the flexibility and the smooth feeling of the hair.

Further, according to the treating method of Examples 1 to 6, the treating agents could be selected to have the compositions as corresponding to the conditions of the hair, thereby giving the softness and evenness of the feelings in combing the treated hair by fingers.

What is claimed is:

1. A method for treating hair, comprising:
applying to hair a hair inside repair agent comprising water and a component to repair an inside of the hair;
applying a nonaqueous pretreatment agent comprising a methylhydrogen polysiloxane, a polyoxyethylene polyoxypropylene alkylether represented by the Formula (1), and an acid, to the hair; and
applying a hair coating agent comprising an amino functional silicone to the hair, 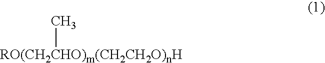
wherein, “R” represents—an alkyl group or alkenyl group having a carbon number of 1 to 24 in which one or more hydrogen atom thereof can be replaced with a substituent, and wherein 1≦m≦20 and 1≦n≦8.

2. A method for treating hair according to claim 1, further applying a film repair agent comprising an amino functional silicone to the hair, so as to repair a film formed on a hair surface derived from the amino functional silicone.

3. A method for treating hair according to claim 1, wherein the amino functional silicone has a structure represented by Formula 2 or Formula 3: 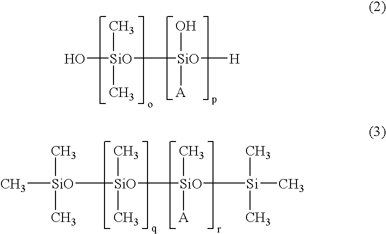
where, “A” represents —(CH₂)₃NH(CH₂)₂NH₂, and where “o” and “p” represent o>1 and p>1 with o+p>10, and where “q” and “r” represent q>1 and r>1 with q+r>10.

4. A method for treating hair according to claim 2, wherein the amino functional silicone has a structure represented by Formula 2 or Formula 3: 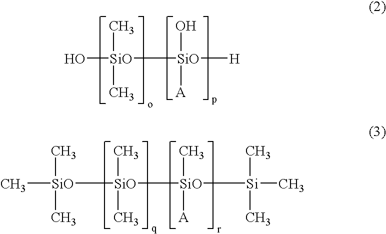
where, “A” represents —(CH₂)₃NH(CH₂)₂NH₂, and where “o” and “p” represent o>1 and p>1 with o+p>10, and where “q” and “r” represent q>1 and r>1 with q+r>10.
